(19) United States
(12) Patent Application Publication
(10) Pub. No.: US 2026/0092030 A1
(43) Pub. Date: Apr. 2, 2026

(54) IMPROVED PROCESS FOR PRODUCING HIGH-PURITY BUTYL ACRYLATE

(71) Applicant: Arkema France, Puteaux (FR)

(72) Inventors: Serge CHOUFFEUR, Saint Avold Cedex (FR); Serge TRETJAK, Saint Avold Cedex (FR)

(21) Appl. No.: 19/120,248

(22) PCT Filed: Oct. 2, 2023
(86) PCT No.: PCT/FR2023/051519

§ 371 Date: Apr. 11, 2025

(30) Foreign Application Priority Data
Oct. 19, 2022 (FR) FR2210802

(51) Int. Cl. C07C 67/08; B01J 27/02; C07C 67/54

(52) U.S. Cl. CPC C07C 67/08; B01J 27/02; C07C 67/54

(57) ABSTRACT

The present invention relates to the production of butyl acrylate by direct esterification of acrylic acid with butanol, this reaction being catalyzed by sulfuric acid. More specifically, it relates to an improved process for producing butyl acrylate, enabling high-purity butyl acrylate to be obtained, by upgrading both the Michael adducts formed during this process, by thermal and catalytic cracking, in the form of reactants and ester which can be recycled in the process, and the final residue, by hydrothermal gasification in the form of methane and mineral salts.

### TECHNICAL FIELD

The present invention relates to the manufacture of butyl acrylate by direct esterification of acrylic acid with butanol, this reaction being catalyzed by sulfuric acid. More specifically, a subject matter of the present invention is an improved process for the manufacture of butyl acrylate, making it possible to obtain high-purity butyl acrylate, by upgrading both the Michael adducts formed during this process, by a thermal and catalytic cracking, in the form of reactants and of esters which can be recycled in the process, and also the final residue, by a hydrothermal gasification, in the form of methane and of inorganic salts.

### TECHNICAL BACKGROUND AND TECHNICAL PROBLEM

The esterification of acrylic acid is an equilibrium reaction with generation of water which it is necessary to remove during the reaction in order to shift the equilibrium in the direction of the production of the acrylic ester.

The problems which are presented during the manufacture of butyl acrylate by direct esterification of acrylic acid, generally in the presence of sulfuric acid as catalyst, are most often related to the complexity of the purification steps necessary after the reaction step in order to obtain a high-purity product, to the detriment of the productive output of the process.

The industrial process, as described in Patent EP 0 609 127 of the applicant company, consists in esterifying acrylic acid (AA) with excess butanol, in the presence of sulfuric acid. The reaction mixture at the end of the reaction comprises butyl acrylate, residual acrylic acid, butyl hydrogen sulfate, traces of sulfuric acid and various impurities resulting from side reactions. This reaction mixture is subsequently subjected to a step of neutralization and of washing with water, the object of which is to remove the “acidic” impurities: residual sulfuric acid, butyl hydrogen sulfate and acrylic acid. This mixture, devoid of acidic impurities, is subjected to various purification steps, which result in the recovery of the purified butyl acrylate. One of the “topping” steps consists in particular in distilling the butanol and the light by-products. The butanol can thus be recycled to the esterification reaction.

The final step of purification of the butyl acrylate consists in sending the mixture containing the ester, freed of the light products, into a last distillation column from which it exits at the top, purified of the heavy by-products which, for their part, are found at the bottom of the distillation column and are subsequently concentrated in an evaporator. The top product from this evaporator, namely the butyl acrylate (BuA), is returned to the bottom of the rectification column, which makes it possible, on the one hand, to recover it as finished product but also to keep the temperature of the column bottom low enough to avoid fouling problems related to the heat-sensitive nature of this monomer.

The residue from the evaporator contains, besides the Michael derivatives and a few percent of free monomers, a few percent of polymerization inhibitors, accumulated during all of the purification steps, such as predominantly phenothiazine in its free form or its form of adduct of AA or of BuA, and also heavy compounds of polymeric nature which are more or less soluble in the medium. In general, this residue is removed by incineration, which results in a significant loss in yield.

Among the by-products generated according to the side reactions, mention may be made of light products, such as butyl acetate, butyl propionate, dibutyl ether or isobutyl acrylate, or heavy products, such as dibutyl maleate.

The “heavy” compounds resulting from Michael addition reactions spontaneously form in units for the production of butyl acrylate. These parasitic reactions are promoted by the high temperatures encountered in particular in the bottoms of distillation columns of these units. Thus, acrylic acid, butanol which has not yet reacted or water of reaction are added to the double bond of butyl acrylate to form mainly:
butyl acryloxypropionate (AA/BuA) by addition of acrylic acid (AA) to butyl acrylate (BuA);
butyl hydroxypropionate (BHP) by addition of water to butyl acrylate;
butyl butoxypropionate (BBP) by addition of butanol to butyl acrylate.

A polyaddition or the formation of mixed compounds is also possible.

One of the characteristics of the heavy by-products is that their boiling point is above the boiling points of acrylic acid, butanol and butyl acrylate. As their volatility is low, they accumulate at the bottom of the last distillation column, at the bottom of the evaporator used to concentrate this residue.

Various solutions have been proposed for the upgrading of these heavy by-products still containing polymerization inhibitors.

The document CN 1063678 proposes methods for the treatment of the oxy-esters formed during the synthesis of butyl acrylate using protic acid catalysts, such as sulfuric acid or para-toluenesulfonic acid. Compounds such as phthalates can also be added, as described in the document U.S. Pat. No. 4,293,347.

The disadvantage of these cracking methods is that the residual product is viscous and contains solids. U.S. Pat. No. 6,617,470 proposes to use, as catalysts, arylsulfonic acids, such as dodecylsulfonic acid, which suppresses the formation of solids in the bottom residue. The document US 2011/0230675 proposes to add water continuously when the cracking is carried out by acid catalysis, in order to avoid the formation of a solid deposit. The document CN 102173990 proposes to add copper salts to the feed of the cracker in order to facilitate the subsequent treatment of the final cracking residue.

Without the applicant company being bound to any one explanation, it believes that it is the interaction between the phenothiazine and the acid catalyst during a catalytic and thermal cracking which is largely responsible for the formation of solid in the final residue of the cracker.

The application FR 2 108 885 employs a thermal cracker without catalyst, which makes it possible to successfully suppress this interaction. However, the cracking yield remains lower than that obtained during a thermal and catalytic cracking.

The application FR 2 111 319 finally provides a simplified process making it possible to carry out a thermal and catalytic cracking while preserving the integrity of the plant by subjecting beforehand the stream from the bottom of the last distillation column to evaporation and the top stream from the latter to two successive condensations. The bottom of this evaporator then contains the heavy residues and the inhibitors and also heavy products of polymeric nature which are more or less soluble. The residue from the cracker will be essentially composed of the unreacted Michael adducts and the acid catalyst used during the cracking.

While the solid deposits in the cracker have been suppressed, it turns out that the step of treatment of the residues, usually carried out by an incineration which upgrades them in the form of heating steam, remains problematic.

In effect, it is possible to opt for two solutions: the two residues, from the bottom of the evaporator and that from the bottom of the cracker, can be treated separately, which results in two operations being carried out, or the two residue streams can be mixed and in fact, on the one hand, the phenothiazine and, on the other hand, the catalyst used during the cracking will be brought together, with as consequence the formation of solids.

The oxidation of organic matter (incineration) to give carbon dioxide and water is a process which has always been known and very often used to treat organic residues and to produce heating steam. In the conventional steam-form energy process, rapid oxidation of organic fuels is often used to produce heat, which is subsequently transferred in a heat exchanger to a fluid, such as water. A heat loss of 10% to 15% is expected as a result of losses necessarily occurring in the exhaust column of conventional boilers. Besides the possible blockage due to solids feeding the boiler, hot spots due to deposits of salts on the boiler tubes or deposits of ash on the faces of the tubes exposed to the flame or to hot gases reduce good heat transmission and consequently the heat transfer efficiency, indeed even cause very costly losses in time as a result of rupture of the walls of the tubes.

From 1981, the document FR 2 481 949 described the oxidation in a supercritical medium, with an additional supply of oxygen in a mixture composed of water and organic inputs, to transform an aqueous stream with a low organic charge into CO₂ and water, thus creating the first incinerator in supercritical condition, more efficient because the combustion energy is conveyed by water under pressure and critical temperature without the need for exchangers, as in conventional boilers.

The document Gazéification de biomasse en eau supercritique [Gasification of Biomass in Supercritical Water] by O. Boutin and J. C. Ruiz, which appeared on May 10, 2013 in the Techniques de l'ingénieur [Techniques of the Engineer] J7010, describes the implementation of hydrothermal gasification to treat certain organic effluents on the pilot scale. This conversion technology makes it possible to convert wet biomass (>70% moisture) into synthesis gas (mixture of methane, hydrogen and carbon dioxide) and to separate the inorganic salts present in the input. It has been employed, for example, in the gasification of algae for the production of hydrogen, the treatment of primary sludges resulting from a sewage treatment plant or the catalytic gasification of pig manure possibly mixed with eucalyptus wood.

There exists a need to have available a process which makes possible the energy upgrading of the final waste, while preventing the formation of solids in the residue which block the incinerators.

It has now been found that, by replacing the evaporator at the bottom of the rectification column and the distillation under reduced pressure and under an inert atmosphere with a single evaporator and its stepped condensation system, the formation of solid in the residue from the cracker is significantly reduced, while simplifying the items of equipment used, in a process making possible the production of high-purity butyl acrylate. In addition, the combination of a hydrothermal gasification process with said evaporator makes it possible to upgrade the final residues in the form of gas with a high calorific value, in particular to give exportable methane gas, instead of converting it into CO₂ by combustion.

### SUMMARY OF THE INVENTION

The present invention makes it possible to meet the abovementioned requirements. More particularly, the invention provides an improved process for the production of butyl acrylate by direct esterification of acrylic acid with butanol which makes it possible to better upgrade the final products usually sent for incineration, by regenerating the starting materials by cracking and by converting the residue into fuel gas. The process according to the invention makes it possible to improve the energy balance of the process while improving the material balance. The invention consists in the implementation of a hydrothermal gasification in combination with the thermal and catalytic cracker used to upgrade the Michael adducts. The object of the invention is to upgrade these adducts as much as possible, in the presence or absence of solid products, on the one hand to give upgradable starting materials recycled in the distillation line and, on the other hand, to generate, in the presence of solids and of salts, a gas phase composed of methane, hydrogen and carbon dioxide, which makes it possible to meet at a minimum the energy requirements of the process and which can be upgraded in the natural gas system of an industrial site or exported.

The invention also applies to an organic product without moisture and not upgraded by hydrothermal gasification until now.

The present invention describes an evaporation system which makes it possible, by the recycling of the butyl acrylate, to maintain a rectification column bottom temperature compatible with the heat-sensitive nature of butyl acrylate and to send the Michael adducts to the thermal and catalytic cracker, while bleeding the very heavy compounds and the polymerization inhibitors at the bottom of this evaporator, for a process making it possible to obtain high-purity butyl acrylate, as described in Patent EP 0 609 127 for the reaction part.

It also complements the purification scheme described in this patent by combining, on the one hand, an evaporator placed at the foot of the column for purification of the butyl acrylate with its stepped condensation system and describes the recycling of the top products resulting from the cracking in the process and, on the other hand, a hydrothermal gasification which makes it possible to transform the residue from the cracking into upgradable gas (methane, hydrogen, CO₂) which makes it possible at a minimum to provide the energy to the process or to upgrade in a gas system.

A subject matter of the invention is a process for the manufacture of butyl acrylate by direct esterification of acrylic acid with excess butanol in the presence of sulfuric acid as catalyst and of at least one polymerization inhibitor, resulting in a crude reaction mixture being obtained which contains butyl acrylate, residual acrylic acid, residual butanol, butyl hydrogen sulfate, traces of sulfuric acid and impurities resulting from side reactions, said process comprising steps of neutralization and of washing with water resulting in a reaction mixture being obtained which is devoid of “acidic” impurities, characterized in that said reaction mixture washed of acidic impurities is subjected at least to the following steps:
a) a topping step in a first distillation column, known as the topping column, making it possible to obtain:
   at the top, a stream composed essentially of unreacted reactants;
   at the bottom, a stream comprising the desired ester and heavy byproducts;
b) a step of rectification by tailing said bottom stream from the topping column in a second distillation column, making it possible to separate:
   at the top, the purified desired ester;
   at the bottom, a stream containing heavy byproducts;
c) a step of concentration of said bottom stream from the rectification column in an evaporator, resulting in a top stream, cooled in two steps, in order to recycle, to the rectification column, the light compounds present and to concentrate the Michael adducts;
d) a step of cracking said concentrate of Michael adducts in a thermal cracker, making it possible to obtain:
   at the top, the noble products resulting from the cracking which are recycled to the feeding of the topping column;
   at the bottom, the final residue,
e) a step of hydrothermal treatment of said residue, in the presence of water, carried out in an item of hydrothermal gasification equipment, resulting in gas of methane, hydrogen and CO₂ type being obtained at the top and in solid residues and water being obtained at the bottom.

The present invention makes it possible to overcome the disadvantages of the state of the art. More particularly, it provides a process which makes it possible to obtain a high-purity butyl acrylate having, as specifications, an ester purity of greater than 99.5%, incorporating an optimized process for the removal of the polymerization inhibitors, making it possible to carry out a cracking of the Michael adducts to give reactants (acrylic acid and alcohol) and to give finished product, thus increasing the productive output of the process and improving the energy balance by the upgrading of the residue to be removed.

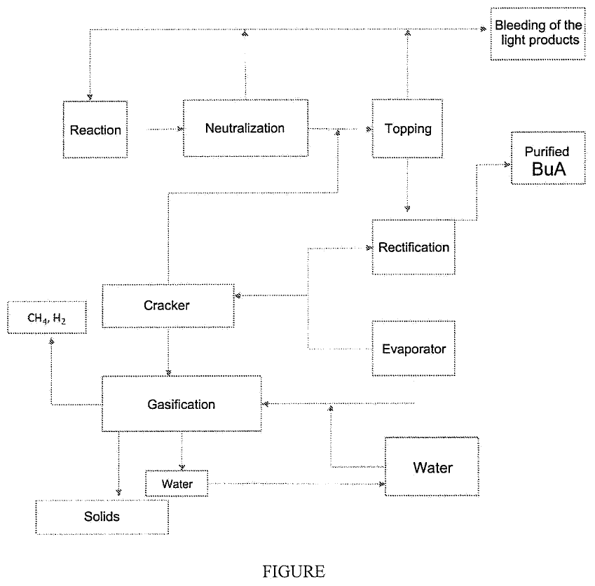

Other characteristics and advantages of the invention will become more apparent on reading the detailed description which follows, with reference to the appended FIG. 1.

FIG. 1: overall diagram of the process for the synthesis of butyl acrylate according to the invention, with the combination of a thermal and catalytic cracker with the items of hydrothermal gasification equipment.

### DETAILED DESCRIPTION OF THE INVENTION

A subject matter of the invention is a process for the manufacture of butyl acrylate by direct esterification of acrylic acid with excess butanol in the presence of sulfuric acid as catalyst and of at least one polymerization inhibitor, resulting in a crude reaction mixture being obtained which contains butyl acrylate, residual acrylic acid, residual butanol, butyl hydrogen sulfate, traces of sulfuric acid and impurities resulting from side reactions.

According to various embodiments, said process comprises the following features, if appropriate combined.

After the esterification step, the process according to the invention comprises steps of neutralization and of washing with water resulting in a reaction mixture being obtained which is freed of the “acidic” impurities: sulfuric acid, butyl hydrogen sulfate, acrylic acid dimer and residual acrylic acid.

Characteristically, the reaction mixture washed of acidic impurities as described above is subjected at least to the following steps:
a) a topping step in a first distillation column, known as the topping column, making it possible to obtain:
   at the top, a stream composed essentially of unreacted reactants;
   at the bottom, a stream comprising the desired ester and heavy byproducts;
b) the bottom stream from the topping column is subjected to a column for rectification by tailing, making it possible to separate:
   at the top, the purified desired ester;
   at the bottom, a stream containing heavy byproducts;
c) a step of concentration of said bottom stream from the rectification column in an evaporator, in particular a thin-film evaporator, resulting in a top stream, cooled in two steps, in order to recycle, to the rectification column, the light compounds present and concentrating the Michael adducts having a very low content of inhibitor;
d) a step of cracking said top stream from the evaporator in a thermal cracker, making it possible to obtain:
   at the top, the noble products resulting from the cracking which are recycled to the feeding of the topping column;
   at the bottom, the final residue,
e) a step of hydrothermal treatment of said residue, in the presence of water, carried out in an item of hydrothermal gasification equipment, resulting in gas of methane, hydrogen and CO₂ type being obtained at the top and in solid residues and water being obtained at the bottom.

According to one embodiment, the thermal cracking may or may not be catalytic.

According to one embodiment, the two residues: from the bottom of the evaporator and the residue from the cracker, are treated separately.

According to one embodiment, the two residues: from the bottom of the evaporator and the residue from the cracker, are mixed and treated in the same gasification operation.

According to one embodiment, said item of hydrothermal gasification equipment comprises a first reactor, a second reactor and a gas-liquid separator.

According to one embodiment, the residue is injected as is into the gasification and the water necessary for the hydrothermal treatment is furthermore injected.

According to one embodiment, the residue is mixed with the water necessary for the hydrothermal treatment before the introduction into the gasification.

According to the embodiment, the hydrothermal gasification is carried out at a temperature of 350° C. to 450° C. and a pressure of 25 MPa.

According to the embodiment, the hydrothermal gasification comprises a gasifier making it possible to separate the salt at the bottom and a gas and liquid mixture at the top.

According to one embodiment, the hydrothermal gasification comprises a separator making it possible to separate the salt under critical conditions, a gasifier and a gas-liquid separator.

According to one embodiment, the concentration of residue/water+residue in the salt separator is of between 10 g/l and 400 g/l.

According to one embodiment, the water used to carry out the hydrothermal gasification can be demineralized water, water resulting from drilling or weakly mineralized water.

According to one embodiment, the water at the outlet of the gasifier, devoid of organic compounds, can advantageously be recycled to the feeding of the separator.

According to one embodiment, the salts obtained and separated can be upgraded as fertilizers.

According to one embodiment, a proportion of 94% to 99% of the carbon introduced into the gasification is upgraded in the form of gas.

According to one embodiment, the gas resulting from the gasification is composed of 40% to 70% of methane, 5% to 20% of hydrogen and 20% to 40% of carbon dioxide.

According to one embodiment, the gases can be further fractionated in order to isolate the methane from the other compounds.

According to one embodiment, the composition by weight of the main compounds of the residue from the bottom of the cracker is as follows:
Butyl butoxypropionate (BBP): 65-90%
para-Toluenesulfonic acid (PTSA): 0.1-4%
Phenothiazine: 100-1000 ppm
Heavy products (molecular weight>264 g/mol): >1%.

According to one embodiment, the composition by weight of the bottom product from the evaporator is as follows:
Butyl butoxypropionate (BBP): 65-85%
para-Toluenesulfonic acid: 0%
Phenothiazine: 1-10%
Heavy products (molecular weight>264 g/mol): >10%.

With reference to FIG. 1, which represents the preferred mode of the invention, the topping section comprises a distillation column having an equivalent of 10 to 30 theoretical stages, preferably 10 to 15 theoretical stages. The internals used for the column can be valve trays or perforated trays having a weir, crossflow trays, such as dual flow trays, ripple trays or Shell Turbogrid trays, or stacked packing, such as structured packing, for example Mellapack 250X from Sulzer.

The topping column is fed in the upper third of this column, preferably between the theoretical stages 3 to 10 counting from the top of the column. The top stream of the column essentially comprises the unreacted reactants. This upgradable stream is recycled to the reaction.

The column operates with a reflux ratio (flow rate of condensed liquid returned to the column/flow rate recycled to the reaction) of between 4/1 and 1/1, preferably 3/1. Advantageously, from 50 to 5000 ppm of polymerization inhibitor are introduced into the purification system according to the process of the invention.

Mention may be made, as polymerization inhibitors which can be used, for example, of phenothiazine (PTZ), hydroquinone (HQ), hydroquinone monomethyl ether (HOME), di(tert-butyl)-para-cresol (BHT), para-phenylenediamine, TEMPO (2,2,6,6-tetramethyl-1-piperidinyloxy), di(tert-butyl)catechol, or TEMPO derivatives, such as OH-TEMPO, alone or their mixtures in all proportions, at contents in the reaction medium which can be of between 50 ppm and 5000 ppm, optionally in the presence of depleted air, but generally at contents of between 150 ppm and 1000 ppm. The addition of the polymerization inhibitors can be carried out at different locations, with the introduction of the reactants or at the top of the distillation column.

In order to make the inhibitors more effective, it is advisable to inject oxygen, air or “depleted” air having 7% O₂ at the bottom of the column. Preferably, the amount of oxygen injected corresponds to a content of 0.2% to 0.5% with respect to the amount of organic vapor in the column.

The column can operate under vacuum, in order to minimize the thermal exposure of the heat-sensitive compounds within the column. Advantageously, the topping column operates under a vacuum ranging from 1000 Pa to 30 000 Pa.

The bottom stream preferably feeds the column making it possible to obtain the purified ester at the column bottom between the theoretical stages 6 to 9.

The column for distillation of the pure product comprises an equivalent of 2 to 15 theoretical stages, preferably 6 to 12 theoretical stages. The internals used for the column can be valve trays or perforated trays having a weir, crossflow trays, such as dual flow trays, ripple trays or Shell Turbogrid trays, or stacked packing, such as structured packing, for example Mellapack 250X from Sulzer.

The top stream from the column consists of high-purity butyl acrylate having, as specifications, an ester purity of greater than 99.5%.

The column operates with a reflux ratio (flow rate of condensed liquid returned to the column/flow rate of pure product) of between ⅛ and 1/1, preferably ¼. Like the topping column, the latter is stabilized and air or depleted air (7% O₂) is injected at the column foot. The column can operate under vacuum, in order to minimize the thermal exposure of the heat-sensitive compounds within the column. Advantageously, the pure product column operates under a vacuum ranging from 1000 pascals to 20 000 pascals.

Advantageously, the operating temperature is between 50° C. and 160° C.

The bottom stream is concentrated on a thin-film evaporator, very well suited to viscous fouling products and to contaminated liquids. The evaporation is carried out in a temperature range from 80° C. to 100° C. and more especially between 90° C. and 100° C. in a pressure range of between 800 Pa and 2000 Pa. The top stream from this evaporator is cooled in two successive steps:
partial condensation in a temperature range from 50° C. to 80° C. and more especially from 60° C. to 70° C. at the same pressure as that of the reactor in order to obtain, on the one hand, a liquid stream of Michael adducts going to feed the cracker and, on the other hand, a vapor stream,
condensation of this vapor stream in a temperature range from 20° C. to 40° C. and more especially from 20° C. to 30° C. before taking up this liquid stream by a pump and mixing with the bottom product from the topping column in order to feed the rectification column.

The bottom residue is subjected, alone or as a mixture, to hydrothermal gasification.

The top adduct stream, and also the para-toluenesulfonic acid catalyst (PTSA) in solution in water or dissolved beforehand in Michael adducts, are continuously introduced into a forced recirculation cracker equipped with an external exchanger in a temperature range from 160° C. to 210° C. a residence time of the order of 2 to 10 h, under a pressure from 200 kPa to atmospheric pressure.

According to one embodiment, the cracking is carried out at a temperature from 160° C. to 180° C. at atmospheric pressure.

The catalyst content, with respect to the amount of adducts feeding the cracker, is of between 0.5% and 3%, such as, for example, in FR 2 901 272. The reactants generated by this cracking, essentially butanol and butyl acrylate, are returned to the purification line of the process. The residue, for its part, is treated, alone or preferably as a mixture, by hydrothermal gasification. The residue and water are injected by two circuits via high-pressure pumps into an item of hydrothermal gasification equipment in a temperature range of between 350° C. and 450° C. and a pressure of 25 MPa.

A first reactor makes it possible to separate the salt at the bottom of the latter from the aqueous organic solution.

A second gasification reactor comprises a catalyst which makes it possible to complete the conversion of the organic products to give gas.

A gas-liquid separator makes it possible to recover, at the bottom, an aqueous phase which can be recycled to the inlet of the separator and a gas phase rich in methane which can be upgraded to produce current which can convey the energy necessary for the operation of the gasification but also for those of the reaction and of the purification line of this process or be furthermore exported. This hydrothermal gasification can be carried out in batch mode or, preferably, in continuous mode.

The examples below illustrate the present invention without, however, limiting the scope thereof.

### EXPERIMENTAL PART

In the examples, the percentages are shown by weight, unless otherwise indicated, and the following abbreviations were used:
PTZ: phenothiazine
BuA: butyl acrylate
BuOH: butanol
BBP: butyl butoxypropionate
DBE: dibutyl ether
BAP: butyl acryloxypropionate
Heavy products: molecular weight>264 g/mol
PTSA: para-toluenesulfonic acid
BPTS: butyl para-toluenesulfononate

### Example 1. Obtaining the Residue Resulting From the Thin-Film Evaporator

A commercially available DV210/1m2 thin-film evaporator heated by 0.4 MPa steam, operating at a pressure of 130 Pa, is fed at a rate of 50 kg/h with a mixture: BuA: 9%; BuOH: 3.5%; BBP: 67%; BAP: 5.7%; PTZ: 1.8%; remainder to 100%: heavy residue.

The top product (40 kg/h) is condensed at 20° C. in a 4 m² tubular exchanger. It is stabilized by addition of a 250 g/h butyl acrylate solution containing 2% of phenothiazine. This top product feeding the cracker comprises 12.5% BuA, 75% BBP, 3% butanol and 900 ppm phenothiazine. The bottom product from the evaporator contains PTZ (6%), approximately 10% of heavy products and BBP as remainder to 100%.

### Example 2. Thermal and Catalytic Cracking Test

A forced recirculation boiler with a volume of 40 1 is used, fed continuously using a diaphragm pump with heavy products/BuA placed on a balance, to which 1% by weight of para- toluenesulfonic acid was added. The feed flow rate is measured using a mass flow meter placed on the feed line and also by the variation in the weight indicated by the balance over time. The operation is carried out at a pressure which is adjusted in order not to vaporize the butyl butoxypropionate. The temperature of the reaction medium and also that at the inlet and at the outlet of the exchanger are continuously measured. The heat-transfer fluid to bring the heat to the exchanger originates from an oil boiler. The heating power is fixed in order to keep the test temperature fixed.

The heavy products/BuA were distilled beforehand under vacuum and contain approximately 600 ppm of phenothiazine.

After cracking at 180° C. under a pressure of 40 kPa (300 mmHg) and a residence time of 9 h, defined as being the volume of the reactor with respect to the feed flow rate by weight of Michael adducts, the top flow rate/feed flow rate ratio is 76% and the top composition comprises 48% of butyl acrylate and 15% of butanol.

The residue, for its part, comprises in particular 0.6% of PTSA, 2.5% of BPTS, 80% of BBP and 10% of heavy products.

### Example 3. Hydrothermal Gasification

The heavy products/BuA mixture is composed of:
Butanol: <0.1%
Butyl acrylate: 5-10%
Butyl hydroxypropionate (BHP): 1-3%
Butyl butoxypropionate (BBP): 70-80%
Butyl acryloxypropionate (BAP): 4-6%
Dibutyl maleate: 2-5%
Phenothiazine: 1-3%.

33 g/h of heavy products/BuA and also 970 g/h of water are introduced via two different pipes into a separator and a catalytic reactor, both operating at 400° C. and 250 bar. After 6 hours of testing under stabilized conditions, the heavy products/BuA are converted into a gas mixture having the following composition by volume: 51% CH₄, 34% CO₂ and 19% H₂. The amount of energy of this gas corresponds to 7096 kWh/ton BuA. The amount of TOC (Total Organic Carbon) is <mg/l.

1. A process for the manufacture of butyl acrylate by direct esterification of acrylic acid with butanol in the presence of sulfuric acid as catalyst and of at least one polymerization inhibitor, resulting in a crude reaction mixture being obtained which contains butyl acrylate, residual acrylic acid, residual butanol, butyl hydrogen sulfate, traces of sulfuric acid and impurities resulting from side reactions, said process comprising steps of neutralization and of washing with water the crude reaction mixture resulting in a reaction mixture being obtained which is devoid of acidic impurities, characterized in that said reaction mixture washed of acidic impurities is subjected to the following steps:
a) a topping step in a first distillation column, known as a topping column to obtain:
a top stream from the topping column composed essentially of unreacted reactants; and
a bottom stream from the topping column comprising a desired ester and heavy byproducts;
b) a step of rectification by tailing said bottom stream from the topping column in a second distillation column, known as a rectification column, to separate:
a top stream from the rectification column containing a purified desired ester; and
a bottom stream from the rectification column containing heavy byproducts;
c) a step of concentrating said bottom stream from the rectification column in an evaporator, resulting in a top stream from the evaporator, and further cooling the top stream from the evaporator in two steps, in order to recycle, to the rectification column, light compounds and to concentrate Michael adducts;
d) a step of cracking said concentrate of Michael adducts in a thermal cracker to obtain:
as a top stream from the thermal cracker, noble products resulting from the cracking which are recycled to the topping column;
a bottom stream from the thermal cracker, a final residue,
e) a step of hydrothermal treatment of said final residue, in the presence of water, carried out in an item of hydrothermal gasification equipment, resulting in gas of methane, hydrogen and CO₂ type being obtained at a top of the item of hydrothermal gasification equipment and in solid residues and water being obtained at a bottom of the item of hydrothermal gasification equipment.

2. The process as claimed in claim 1, in which said acidic impurities are sulfuric acid, butyl hydrogen sulfate, acrylic acid dimer and residual acrylic acid.

3. The process as claimed in claim 1, in which the polymerization inhibitor is chosen from: phenothiazine (PTZ), hydroquinone (HQ), hydroquinone monomethyl ether (HQME), di(tert-butyl)-para-cresol (BHT), para-phenylenediamine, TEMPO (2,2,6,6-tetramethyl-1-piperidinyloxy), di(tert-butyl)catechol, OH-TEMPO, or their mixtures in all proportions.

4. The process as claimed in claim 1, in which the polymerization inhibitor is used in the reaction medium or in the purification steps at contents of between 50 ppm and 5000 ppm.

5. The process as claimed in claim 1, in which the polymerization inhibitor is added at different locations, with the reactants or at the top of the distillation column.

6. The process as claimed in claim 1, in which the step of concentrating in an evaporator is carried out in a temperature range from 80° C. to 100° C., and at a pressure of between 800 Pa and 2000 Pa.

7. The process as claimed in claim 1, in which the top stream from the evaporator is cooled in two successive steps:
a. partial condensation in a temperature range from 50° C. to 80° C. at the same pressure as that of the evaporator in order to obtain, on the one hand, a liquid stream of Michael adducts, having a content of inhibitors of less than 1000 ppm, going to feed the cracker and, on the other hand, a vapor stream; and
b. condensation of said vapor stream in a temperature range from 20° C. to 40° C. before taking up a liquid stream obtained from said condensation by a pump and feeding to the rectification column.

8. The process as claimed in claim 1, in which the cracking is carried out at a temperature from 160° C. to 180° C. at atmospheric pressure.

9. The process as claimed in claim 1, in which the butyl acrylate obtained has a purity of greater than 99.5%.

10. The process as claimed in claim 1, in which the hydrothermal gasification comprises a separator to separate a salt under critical conditions, a gasifier and a gas-liquid separator.

11. The process as claimed in claim 10, in which a concentration of residue/water+residue in said separator is of between 10 g/l and 400 g/l.

12. The process as claimed in claim 1, in which the step of hydrothermal treatment of said final residue generates a gas composed of 40% to 70% of methane, 5% to 20% of hydrogen and 20% to 40% of carbon dioxide.

13. The process as claimed in claim 10-one of claims 10-to 12, in which water at an outlet of the gasifier, devoid of organic compounds, is recycled to the separator.

14. The process as claimed in claim 6, in which the temperature range is from 90° C. to 100° C.

15. The process as claimed in claim 7, in which the temperature range of the partial condensation is from 60° C. to 70° C.

16. The process as claimed in claim 7, in which the temperature range of the condensation of said vapor stream is from 20° C. to 30° C.
